SEQUENCE LISTING (8 sequences)

SEQ ID NO: 1 - protein, 37 aa, Artificial Sequence
  Synthetic
KCNTATCATQRLANFLVHSSNNFGAILSSTNVGSNTX

SEQ ID NO: 2 - protein, 37 aa, Artificial Sequence
  Synthetic
KCNTATCATQRLANFLVHSSNNXGAILSSTNVGSNTY

SEQ ID NO: 3 - protein, 37 aa, Artificial Sequence
  Synthetic
KCNTATCATQRLANXLVHSSNNFGAILSSTNVGSNTY

SEQ ID NO: 4 - protein, 37 aa, Artificial Sequence
  Synthetic
KCNTATCATQRLANFLVHSSNNXGAILSSTNVGSNTX

SEQ ID NO: 5 - protein, 37 aa, Artificial Sequence
  Synthetic
KCNTATCATQRLANXLVHSSNNXGAILSSTNVGSNTY

SEQ ID NO: 6 - protein, 37 aa, Artificial Sequence
  Synthetic
KCNTATCATQRLANXLVHSSNNFGAILSSTNVGSNTX

SEQ ID NO: 7 - protein, 37 aa, Artificial Sequence
  Synthetic
KCNTATCATQRLANXLVHSSNNXGAILSSTNVGSNTX

SEQ ID NO: 8 - protein, 37 aa, Homo sapiens [sequence omitted]

(19) United States
(12) Patent Application Publication
(10) Pub. No.: US 2010/0021390 A1
(43) Pub. Date: Jan. 28, 2010

(54) Fluorescent Analogs Of The Islet Amyloid Polypeptide

(75) Inventors: Daniel P. Raleigh, Stony Brook, NY (US); Andisheh Abedini, Jefferson City, MO (US); Peter Marek, St. James, NY (US); Ruchi Gupta, New York, NY (US); Fanling Meng, Stony Brook, NY (US)

Correspondence Address: Peter G. Carroll;MEDLEN & CARROLL, LLP, Suite 350, 101 Howard Street, San Francisco, CA, 94105, US

(73) Assignee: The Research Foundation of State University of New York

(21) Appl. No.: 12/405,519

(22) Filed: Mar. 17, 2009

Related U.S. Application Data
(60) Provisional application No. 61/037,233, filed on Mar. 17, 2008.

(51) Int. Cl. A61K 49/00; C07K 14/00; G01N 33/00; G01N 33/53

(52) U.S. Cl. 424/9.6; 530/324; 436/87; 435/7.1

(57) ABSTRACT

The present invention relates to methods and compositions for the investigation of amyloid formation. In preferred embodiments, amyloid formation associated with type II diabetes is monitored by fluorescence spectroscopic measurements of the activity of p-cyano-phenylalanine-substituted islet amyloid polypeptide, and derivatives thereof, under an amyloid-forming condition. In some embodiments, the amyloid-forming condition is associated with a diseased, or putatively diseased, cell or tissue. In some embodiments, the invention provides a method of evaluating the severity of the amyloid-forming condition, and changes therein induced by agents that inhibit, or potentially inhibit, amyloid formation.

### STATEMENT OF GOVERNMENT SUPPORT

This invention was made in part with government support under grant numbers GM-070941 from the National Institutes of Health and PRF 44740-AC4 from the American Chemical Society Petroleum Research Fund. As such, the United States government has certain rights to the invention.

### FIELD OF THE INVENTION

The present invention relates to methods and compositions for the investigation of amyloid formation. In preferred embodiments, the invention relates to compositions comprising the islet amyloid polypeptide and derivatives thereof. In some embodiments, the invention relates to the investigation of diseases and disorders associated with amyloid formation using the methods and compounds disclosed herein. In some embodiments the invention relates to the screening of candidate drugs that prevent or inhibit amyloid formation.

### BACKGROUND OF THE INVENTION

Amyloids are insoluble, fibrous protein and/or peptide aggregations characterized by a cross-beta sheet quaternary structure. The formation of amyloid from one or another monomer species has been implicated in more than twenty different human diseases including Alzheimer's disease, Parkinson's disease, prion-based diseases and type II diabetes. In the latter, the peptide thought to serve as the major constituent of amyloid is the islet amyloid polypeptide (IAPP). IAPP, also called amylin, is an endocrine hormone secreted by the pancreas. IAPP is 37 residues in length and contains a disulfide bridge between Cys-2 and Cys-7. The C-terminus of the hormone is amidated.

Unfortunately, few if any spectroscopic methods lend themselves to examining the process of amyloid formation. In particular, information relevant to interactions among individual amino acid residues of amyloid forming proteins and/or peptides is generally not obtainable. The most familiar means of monitoring amyloid formation involves employing fluorescence measurements using thioflavin-T, a fluorescent dye that binds to amyloid. Its fluorescence increases when it binds to formed amyloid, providing a measure of the presence or absence of formed amyloid. The dye generally does not bind to individual elements such as monomeric proteins or intermediates that comprise the amyloid, however. Thus, the fluorescent signal from thioflavin-T cannot be interpreted as a marker of any particular constituent of the formed amyloid. Accordingly, the molecular origins of the increased fluorescence are not rigorously understood, although models suggest that the dye binds in grooves on the surface of amyloid. Moreover, some compounds can displace the bound dye and thus be mistaken for inhibitors of amyloid formation. Spectroscopy is involved in measuring the circular dichroism of formed amyloid, but circular dichroism also provides information only about formed amyloid. Thus, an improved method for spectroscopically monitoring the formation of amyloid in the presence of IAPP is needed.

### SUMMARY OF THE INVENTION

The present invention relates to methods and compositions for the investigation of amyloid formation. In preferred embodiments, the invention relates to compositions comprising the islet amyloid polypeptide and derivatives thereof. In some embodiments, the invention relates to the investigation of diseases and disorders resulting from amyloid formation using the methods and compounds disclosed herein. In some embodiments the invention relates to the screening of candidate drugs that prevent amyloid formation.

In some embodiments, the invention relates to a composition selected from the group consisting of:

KCNTATCATQRLANFLVHSSNNFGAILSSTNVGSNTF_{C≡N}
(“IAPP-Y37F_{C≡N}”),
KCNTATCATQRLANFLVHSSNNF_{C≡N}GAILSSTNVGSNTY
(“IAPP-F23F_{C≡N}”),
KCNTATCATQRLANF_{C≡N}NLVHSSNNFGAILSSTNVGSNTY
(“IAPP-F15F_{C≡N}”),
KCNTATCATQRLANFLVHSSNNF_{C≡N}GAILSSTNVGSNTF_{C≡N}
(“IAPP-F23, Y37F_{C≡N}”),
KCNTATCATQRLANF_{C≡N}LVHSSNNF_{C≡N}GAILSSTNVGSNTY
(“IAPP-F15, F23F_{C≡N}”),
KCNTATCATQRLANF_{C≡N}LVHSSNNFGAILSSTNVGSNTF_{C≡N}
(“IAPP-F15, Y37F_{C≡N}”),
and
KCNTATCATQRLANF_{C≡N}LVHSSNNF_{C≡N}GAILSSTNVGSNTF_{C≡N}
(“IAPP-F15, 23, 37F_{C≡N}”)

wherein F_{C≡N} is p-cyanophenylalanine.

In some embodiments, the invention relates to a method of monitoring a process of amyloid formation in vitro comprising: (i) providing a) a fluorescence spectrometer, and b) a composition comprising an islet amyloid polypeptide having a p-cyanophenylalanine substitution, (ii) creating an in vitro condition in which amyloid forms, (iii) adding said substituted polypeptide to said amyloid-forming condition to make a mixture and (iv) detecting a fluorescence from said mixture.

In one embodiment, said mixture is made from an amyloid-forming condition created in a solution comprising said polypeptide.

In some embodiments, said in vitro condition comprises a tissue. In some embodiments, said in vitro condition comprises a cell. In some embodiments said cell is a cultured cell. In further embodiments, said cultured cell comprises a primary cell culture. In still further embodiments, said cell is a transformed cell. In additional embodiments, said cell is a stem cell. In some embodiments, said tissue comprises a tissue culture. In still further embodiments, said tissue is a biopsied tissue. In additional embodiments, said biopsied tissue is from a tissue for transplantation. In preferred embodiments, said tissue is a mammalian tissue. In some embodiments, said cell is a mammalian cell. In further embodiments, said mammalian tissue or cell is from a mammal suspected of having type II diabetes.

In some embodiments, said mixture further comprises a test compound. In some such embodiments, said test compound is added before said substituted amyloid polypeptide is added. In others, said test compound is added after said substituted amyloid polypeptide is added.

In further embodiments, the invention relates to a method of monitoring a process of amyloid formation in an organism in vivo comprising: (i) providing a fluorescence spectrometer, (ii) administering to said organism an islet amyloid polypeptide having a p-cyanophenylalanine substitution to create a treated organism, and (iii) detecting a fluorescence in or from said treated organism. In some embodiments, said fluorescence is from a cell or tissue removed from said treated organism. In some embodiments, said treated organism is additionally treated with a test compound. Said test compound treatment may precede or succeed said administration of said substituted amyloid polypeptide.

In some embodiments, the invention relates to a method of monitoring a process of amyloid formation comprising: (i) providing a) a fluorescence spectrophotometer, b) a composition comprising an islet amyloid polypeptide having a p-cyanophenylalanine substitution, and c) a condition in which amyloid forms, (ii) adding said substituted polypeptide to said condition, (iii) removing from said condition a sample of said condition, and (iv) detecting a fluorescence from said sample.

In some embodiments, the invention relates to a method of monitoring a process of amyloid formation comprising: (i) providing a) a fluorescence spectrophotometer, b) a composition comprising an islet amyloid polypeptide having a p-cyanophenylalanine substitution, and c) a condition in which amyloid forms, (ii) adding said composition to said condition, and (iii) detecting a fluorescence from said condition.

In some embodiments, said condition is in vivo. In some embodiments, said condition is in vitro. In some embodiments, said condition comprises a cell. In further embodiments, said condition comprises a tissue. In some embodiments, said cell comprises a primary cell culture. In still other embodiments, said cell is a transformed cell. In additional embodiments, said cell is a stem cell. In some embodiments, said tissue comprises a tissue culture. In still further embodiments, said tissue is a biopsied tissue. In additional embodiments, said biopsied tissue is from a tissue for transplantation.

### BRIEF DESCRIPTION OF THE DRAWINGS

For a more complete understanding of the features and advantages of the present invention, reference is now made to the detailed description of the invention along with the accompanying figures.

FIG. 1A shows a schematic representation of the cross-β geometry of amyloid fibrils, adapted from Dobson, Seminars in Cell & Developmental Biology 15, 3-16 (2004), incorporated herein by reference. The diagram depicts four protofibrils. Each protofibril is made up of two long β-sheets. The polypeptide chain runs perpendicular to the fibril axis. FIG. 1B shows a representation of the progress of fibril formation. The solid line represents an unseeded reaction, in other words a reaction that starts from monomeric proteins or peptides. A distinct lag phase is observed followed by a rapid growth phase. It has been observed that seeding (dashed line) may surpass the lag phase. Seeding refers to the addition of pre-formed amyloid.

FIG. 2A shows a comparison of the time course for thioflavin-T fluorescence for wild-type hIAPP () and hIAPP-Y37F_{C≡N} (∘). The data is normalized on the vertical axis to a scale from 0 to 1.0. FIG. 1B shows a transmission electron microscopy (TEM) image of the fibrils formed by wild-type human IAPP, while FIG. 2C shows a transmission electron microscopy (TEM) image of the hIAPP-Y37F_{C≡}N variant fibrils. The scale bars in the lower right hand corners of FIGS. 1B and 1C represent a length of 100 nm. The experiments were performed in 2% HFIP and 20 mM Tris-HCl, pH 7.4.

FIG. 3A shows a circular dichroism spectrum for hIAPP-Y37F_{C≡N} recorded at the end of the fibrillization reaction. FIG. 3B shows a circular dichroism spectrum for wild-type hIAPP. Experiments were performed in 2% HFIP and 20 mM Tris-HCl, pH 7.4.

FIG. 4A shows a p-cyanophenylalanine fluorescence emission spectra of hIAPP-Y37F_{C≡N} at the start of the fibrillization reaction (e) and at the end of the reaction (∘). Experiments were performed in 2% HFIP and 20 mM Tris-HCl, pH 7.4. FIG. 4B shows a comparison of the time course of thioflavin-T fluorescence () at 480 nm and p-cyanophenylalanine fluorescence (o) at 296 nm for hIAPP-Y37F_{C≡N} at pH 7.4, 25° C. The vertical axis is normalized so that the total signal change is displayed on a scale of 0.0 to 1.0. The molecular representation of p-cyanophenylalanine is shown as an insert.

FIG. 5A shows an emission spectrum for human IAPP containing the F15F_{C≡N} amino acid substitution. FIG. 5B shows an emission spectrum for human IAPP containing the F23F_{C≡N} amino acid substitution. FIG. 5C shows an emission spectrum for human IAPP containing the Y37F_{C≡N} amino acid substitution. The closed circles () represent the experiment conducted in the absence of rifampicin. Open circles (∘) correspond to an experiment conducted in the presence of 15 μM rifampicin. The stars indicate the time points at which aliquots were removed for TEM.

FIG. 6A shows a transmission electron microscopy (TEM) image of the hIAPP-F15F_{C≡N} fibrils. FIG. 6B shows a transmission electron microscopy (TEM) image of the hIAPP-F23F_{C≡N} fibrils. FIG. 6C shows a transmission electron microscopy (TEM) image of the hIAPP-Y37F_{C≡N} fibrils. The scale bars in the lower right hand corners of each of the figures represent a length of 100 nm. The experiments were performed in 2% HFIP and 20 mM Tris-HCl, pH 7.4.

FIG. 7 depicts schematically a possible mechanism for amyloid formation. It is not intended that the present invention be limited to any particular mechanism for amyloid formation. Monomers associate to form oligomeric species, which then assemble to ‘seed’ rapid growth. Secondary nucleation is dependent on the presence of fibrils and or protofibrils.

FIG. 8A shows a time course experiment demonstrating amyloid formation as monitored by thioflavin-T fluorescence assay for hIAPP-F15F_{C≡N} (closed circles) and by p-cyanoPhe fluorescence (open circles). The data is normalized so that the total signal change is displayed on a scale of 0.0 to 1.0. The molecular representation of 4-cyanophenylalanine is shown as an insert. FIG. 8B shows a time course experiment demonstrating amyloid formation as monitored by thioflavin-T fluorescence assay for hIAPP-F23F_{C≡N} (closed circles) and by p-cyanoPhe fluorescence (open circles). The data is normalized so that the total signal change is displayed on a scale of 0.0 to 1.0. The molecular representation of 4-cyanophenylalanine is shown as an insert. FIG. 8C shows a time course experiment demonstrating amyloid formation as monitored by thioflavin-T fluorescence assay for hIAPP-Y37F_{C≡N} (closed circles) and by p-cyanoPhe fluorescence (open circles). The data is normalized so that the total signal change is displayed on a scale of 0.0 to 1.0.

FIG. 9 shows a molecular representation of rifampicin.

FIG. 10A shows thioflavin-T fluorescence monitored assays of fibril formation. The closed circles () represent the experiment conducted in the absence of rifampicin. Open circles (◯) correspond to an experiment conducted in the presence of 15 μM rifampicin. The stars (⋆) indicated the time points at which aliquots were removed for TEM. FIG. 10B shows a TEM image of the fibrillization reaction product for IAPP without rifampicin. FIG. 10C shows a TEM image of a sample of 32 μM peptide and 15 μM rifampicin collected at 100 minutes after the start of the fibrillization reaction. Reactions were conducted at 25° C., pH 7.4, 32 μM IAPP, 25 μM thioflavin-T in 2% HFIP. Scale bar is 100 nm.

FIG. 11A shows a thioflavin-T fluorescence monitored time course assay of IAPP fibril formation. Rifampicin was added at the point indicated by the arrow (↓). FIG. 11B shows a TEM image recorded before the drug was added at a time indicated by the (◯). FIG. 11C shows a TEM image recorded 35 minutes after addition of the drug, indicated by the (⋆). All samples were 32 μM IAPP, 25 μM thioflavin-T in 2% HFIP, 25° C., pH 7.4. Scale bar is 100 nm.

FIG. 12A shows thioflavin-T fluorescence monitored assays of fibril formation. The closed circles () represent the experiment conducted in the absence of rifampicin. Open circles (◯) correspond to an experiment conducted in the presence of 15 μM rifampicin. The stars (⋆) indicated the time points at which aliquots were removed for TEM. FIG. 12B shows a TEM image of the product of the fibrillization reaction for IAPP without rifampicin. FIG. 12C shows a TEM image of a sample of 32 μM peptide and 15 μM rifampicin collected at 110 minutes after the start of the fibrillization reaction. Reactions were conducted at 25° C., pH 7.4, 32 μM IAPP, 83 mM ascorbic acid, 25 μM thioflavin-T in 2% HFIP. Scale bar is 100 nm.

FIG. 13A shows a thioflavin-T fluorescence monitored time course assay of IAPP fibril formation. Rifampicin was added at the point indicated by the arrow (↓).

FIG. 13B shows a TEM image recorded 35 minutes after addition of the drug, indicated by the (⋆). All samples were 32 μM IAPP, 83 mM ascorbic acid, 25 μM thioflavin-T in 2% HFIP, 25° C., pH 7.4. Scale bar is 100 nm.

FIG. 14A shows a thioflavin-T fluorescence monitored time course assay of IAPP fibril formation with or without rifampicin. The closed circles () represent the experiment conducted in the absence of rifampicin. Open circles (◯) correspond to an experiment conducted in the presence of 15 μM rifampicin. Rifampicin in Tris-HCl buffer, pH 7.4 was incubated for 35 days at room temperature before the start of the experiment. All samples were 32 μM IAPP, 25 μM thioflavin-T in 2% HFIP, 25° C., pH 7.4. FIG. 14B shows a TEM image of a sample collected at the indicated time point (⋆) for a sample of IAPP without rifampicin. FIG. 14C shows a TEM image of a sample collected at the indicated time point (◯) for a sample of IAPP plus rifampicin. Scale bar is 100 nm.

FIG. 15A shows fluorescence-detected kinetics of IAPP Y37FCN in the absence of rifampicin. FIG. 15B shows fluorescence-detected kinetics of IAPP Y37FCN in the presence of rifampicin (15 μM). All samples were 32 μM peptide, 25 μM thioflavin-T in 2% HFIP, 25° C., pH 7.4. Excitation was at 240 nm and the emission was monitored at 296 nm.

### DEFINITIONS

To facilitate the understanding of this invention, a number of terms are defined below. Terms not specifically defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “amyloid” refers to an aggregation of peptides and/or proteins into insoluble, fibrous forms. The terms “amyloid” and “amyloid fibril” are used interchangeably herein. In a preferred embodiment, the peptide is the islet amyloid polypeptide. “Intermediate amyloid” or “amyloid intermediates” refer to protein- and/or peptide-based compositions that ultimately give rise to but have yet to form the polymeric, cross-p-sheet structured plaques that, in vivo, are deposited on and around organs and nerves, and are referred to herein as “mature plaque.” The general term “plaque” encompasses any polypeptide, in vivo or in vitro, whose polymeric state assumes a cross-beta pattern, including but not limited to plaque identified by traditional tests such as the congo red birefringence test. The term “protofilament” also refers to a cross-β-sheet structure. The term “insoluble” in reference to plaque herein is not intended to limit the meaning of plaque to a population of peptides trapped in an absolutely insoluble state.

The term “fibrillization reaction” refers to the process of amyloid formation. The term “fibrillization” refers to the process of amyloid formation. The term “amyloid formation reaction” refers to the process of amyloid formation. The term t₅₀ refers to the time required for the amyloid formation reaction to go to 50% completion. As used herein, an “amyloid forming condition” refers to any condition or set of conditions, whether in vitro or in vivo, that promotes the eventual formation of mature amyloid plaque. While it is not intended that the present invention be limited to any particular mechanism or conditions under which amyloid forms, conditions that may potentially contribute to amyloid formation include but are not limited to the misfolding of proteins and/or peptides capable of forming amyloid, premature cell death/apoptosis, incorrect processing of proteins and/or peptides and ionic imbalance in affected cells or tissue.

As used herein, “islet amyloid polypeptide,” “IAPP” and “amylin” refer, interchangeably, to a peptide secreted by pancreatic β-cells. IAPP comprises 37 residues; the wild-type human form of IAPP (hIAPP) has the amino acid sequence KCNTATCATQRLANFLVHSSNNFGAILSSTNVGSNTY (n-terminus at lysine (“K”), c-terminus at tyrosine (“Y”)) wherein the cysteine (“C”) residues may optionally be connected to one another by a disulfide bridge and the c-terminus is preferably amidated. The several p-cyanophenylalanine-substituted IAPP molecules claimed herein are abbreviated as IAPP followed by single letter code for the amino acid(s) substituted, its numbered position in the sequence and the abbreviation “F_{C≡N}” to denote p-cyanophenylalanine.

A “cross-β-sheet structure” refers to a structure in proteins in which a stretch of amino acids connect laterally to form hydrogen bonds and give rise to a twisted, “sheet-like” quaternary structure.

“Hydrophobicity” refers to a physical property of a molecule characterized by the tendency of the molecule to be repelled from a mass of water.

“Solvent interactions” refer to influences of a solvent or solvents on the kinetics and/or thermodynamics of a reaction.

As used herein, the term “p-cyanophenylalanine” refers to the amino acid phenylalanine having at its para or 4-position the cyano chemical substituent in accordance with conventional chemical nomenclature. Thus, the compound may also be referred to as 4-cyanophenylalanine. The compound has the following general chemical structure:

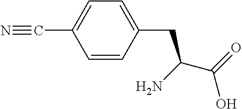

As used herein, “type II (or type-2) diabetes” or “type II diabetes mellitus” refers to a syndrome characterized by disordered metabolism and inappropriately high blood sugar (hyperglycemia) resulting from abnormal resistance to insulin's effects coupled with, in some stages of the disease, inadequate levels of insulin secretion. Examples of symptoms associated with diabetes include but are not limited to increased thirst and appetite, dry mouth, frequent urination, fatigue, blurred vision, headaches and unexplained weight loss.

“Alzheimer's disease” refers to a neurodegenerative, progressive and terminal disease that commonly causes dementia due to cognitive deterioration. Symptoms include but are in no way limited to memory loss, confusion, anger, language breakdown and mood swings.

“Parkinson's disease” refers to a degenerative disorder of the central nervous system in which the patient's motor skills and speech are adversely affected. Symptoms of Parkinson's disease include but are in no way limited to tremors, muscular rigidity, bradykinesia and postural instability.

“Amyloidosis” refers to a condition whereby amyloid proteins and/or peptides are abnormally deposited in organs and/or tissues. The condition contributes to type-2 diabetes and results in the onset of diseases that include but are not limited to Alzheimer's disease, Parkinson's disease and inclusion body myositis.

The term “fluorescence” refers generally to the optical phenomenon in which molecular absorption of a photon triggers the emission of another photon with a longer wavelength. Use of the term, however, is not intended to exclude phenomena such as multi-photon fluorescence or upconversion (wherein the emitted light is at a higher energy than the absorbed photons). Objects and entities that exhibit this property are said to be “fluorescent”. A myriad of technologies and detection methods are used to observe and/or measure fluorescence, which include but are in no way limited to fluorescence resonance energy transfer (FRET) applications, fluorescence emission spectroscopy, fluorescence microscopy, DNA microarrays, fluorescence assisted cell sorting (FACS) and high-performance liquid chromatography (HPLC) assisted array detection.

“Subject” or “test subject” refer to any mammal, preferably a human patient, livestock, or domestic pet.

The term “cell” as used herein encompasses any one of the smallest units of an organism, which units can be classified as living. Some embodiments of the invention can be practiced using cells that are no longer living, or using cells that have been removed from the organism. The latter, if alive in vitro, comprise a “cell culture.” A non-limiting example of the former is a histological section. A “primary cell” may refer to a cell in an organism or removed (isolated, explanted) from an organism, whether or not the cell is descended from a cell that has undergone division in vitro, so long as its genome has not changed from that of its in vivo ancestor. Division of such cells in vitro results in “primary cell cultures.” “A transformed cell,” which may arise in vivo or in vitro, has acquired foreign genetic material and thus has a genome that differs from that of its ancestral line. A “stem” cell herein refers broadly to any cell that is capable of division and differentiation to a phenotype different in some respect from its ancestral line.

A “tissue” encompasses any plurality of interconnected cells wherein a biological function inheres in the interconnection.

A “tissue culture” may be synonymous with “cell culture” herein or, when the context so admits, with “tissue,” provided only that the tissue has been removed from the organism. In some embodiments, cells comprising the tissue may be no longer alive, or alive but not dividing, or alive and dividing. The tissue may comprise more than one tissue type and/or more than one cell type as is typical of biopsied tissue, for example.

“Transplantation” as used herein encompasses the transplantation of cells, tissues or organs from one site to another) within a single organism (“autotransplantation” or from one individual to another, without intending any limitation as to immunological compatibility.

The “single letter amino acid code” refers to the designation of each of the twenty naturally occurring amino acids using a one-letter designation. The code is as follows for each of said twenty naturally occurring amino acids: G=glycine, A=alanine, L=leucine, M=methionine, F=phenylalanine, W=tryptophan, K=lysine, Q=glutamine, E=glutamic acid, S=serine, P=proline, V=valine, I=isoleucine, C=cysteine, Y=tyrosine, H=histidine, R=arginine, N=asparagine, D=aspartic acid, T=threonine.

As used herein, “quantum yield” is a radiation-induced process by which the efficiency of light absorbed by an entity generates an intrinsic effect. In entities that exhibit fluorescent properties, the “fluorescence quantum yield” refers to the ratio of the number of photons emitted to the number of photons absorbed by said entity.

The term “test compound” refers to any molecule that might be useful as an inhibitor of amyloid formation. Embodiments of the invention enable candidate compounds to be screened rapidly.

### DETAILED DESCRIPTION OF THE INVENTION

The present invention relates to methods and compositions for the investigation of amyloid formation. In preferred embodiments, the invention relates to compositions comprising the islet amyloid polypeptide and derivatives thereof. In some embodiments, the invention relates to the investigation of diseases and disorders resulting from amyloid formation using the methods and compounds disclosed herein. In some embodiments the invention relates to the screening of candidate drugs that prevent amyloid formation.

The formation of amyloid is the pathological marker in more than twenty different human diseases including Alzheimer's disease and type-2 diabetes. Embodiments of the present invention relate especially to amyloid formation by islet amyloid polypeptide (IAPP), also known as amylin, the endocrine hormone responsible for pancreatic islet amyloid in type II diabetes. Although applicants will not be bound anywhere herein by any theory that seeks to explain how or by what mechanisms any aspect of the invention works, it is generally accepted that islet amyloid contributes directly to the pathology of type 2 diabetes by contributing to β-cell dysfunction and death: islet amyloid appears to accelerate the loss of β-cell mass and to promote a characteristic decline in insulin production and secretion as described in Ritzel et al. Diabetes 56, 65-71 (2007), hereby incorporated by reference. Recently, islet amyloid has emerged as a major problem in islet cell transplantation as provided in Cardona et al., Nature Medicine 12, 304-306 (2006), hereby incorporated by reference. Despite these indictments, comparatively little is known about how amyloid forms in the presence of IAPP. Embodiments of the present invention, by providing fine-textured insight into the process of amyloid formation, are expected to sharpen the search for inhibitors of IAPP-based amyloid formation.

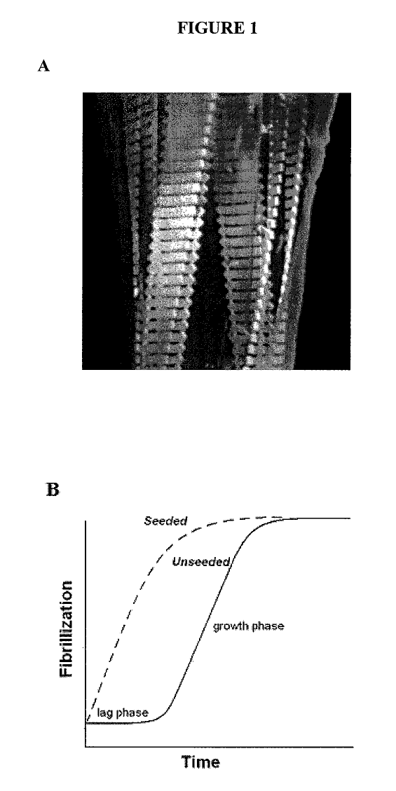

Amyloid is a generic term referring to deposits of insoluble, proteinaceous aggregates in tissue. A large number of proteins and polypeptides that do not form amyloid in vivo can be induced to do so in vitro. Despite the considerable structural and amino acid sequence diversity amongst the proteins known to form amyloid, all amyloid fibrils share several common morphological features. Amyloid fibrils are typically unbranched and are 5 to 10 nm in diameter, giving rise to characteristic transmission electron microscopy (TEM) images as exemplified in Sunde et al., Journal of Molecular Biology 273, 729-739 (1997), hereby incorporated by reference. A defining feature of the amyloid fibril is the cross-β-sheet motif. In this motif, β-sheets extend over the length of the fibril with the individual β-strands aligned perpendicular to the fibril axis as depicted in FIG. 1A.

Individual fibrils are built up from protofilaments. The kinetics of amyloid formation are complex and typically exhibit a lag phase followed by a much more rapid growth phase (also called the elongation phase) as shown in FIG. 1B. The lag phase can often be abolished by seeding a solution of unaggregated peptide with a small amount of pre-formed fibrils as described in Harper et al. Annual Review of Biochemistry 66, 385-407 (1997), hereby incorporated by reference. Seeding is generally, although not completely, specific. Fibrils are often, but not always, best at seeding reactions that contain the same monomers from which they were formed. This indicates that there must be some differences in the structure of the amyloid fibrils derived from different proteins even though their secondary structure and ultrastructure appear similar.

As amyloid forms, the ordering of the amyloid fibrils promotes binding of dyes such as thioflavin-T and Congo Red. In exploitation of this property of amyloid, such dyes are commonly used as markers of amyloid formation. Thioflavin-T, which fluoresces at increased intensity when bound, is a standard means for monitoring rates of formation of amyloid in solution. As is detailed in the Examples herein, however, the approach has faults not shared by preferred embodiments of the present invention.

In such preferred embodiments, the invention relates to the measurement of the properties of a p-cyanophenylalanine substituted islet amyloid polypeptide using a fluorescence spectrometer. Properties such as IAPP's propensity to polymerize, to form helical intermediates, to aggregate and/or to bind to aberrant variants of IAPP or to elements of the extracellular matrix, are among the properties amenable to investigation with the preferred embodiments. These properties tend to manifest themselves in seriatim as different amyloid morphologies that, taken together, describe a process that is indirectly observable as changes in excited fluorescence. An agent that significantly affects the kinetics of the process is a putative drug, assuming that the test agent only changes the kinetics and does not interfere with the assay.

Compounds (including aromatic residues in proteins) that interfere with the fluorescent signal in the standard thioflavin-T assay do not interfere in embodiments of the present invention. A blue-shifted absorption band, i.e. a shift to the higher energy portion of the electromagnetic spectrum, in cyanophenylalanine's spectrum allows the artisan to excite fluorescence without at the same time stimulating background fluorescence in the presence of other proteins that contain inherently fluorescent amino acids. Moreover, cyanophenylalanine becomes part of a molecule that gets incorporated into amyloid. Thioflavin-T does not. Thioflavin-T merely binds to such molecules in an undefined way, with no guarantee that it fails to bind to molecules not involved in amyloid formation. In addition, there is no guarantee that potential inhibitors of amyloid formation will not displace thioflavin-T from amyloid. If this were to occur it would lead to false positives in an amyloid inhibition assays which used thioflavin-T florescence.

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); hIAPP (human islet amyloid polypeptide); Fmoc (9H-fluoren-9-ylmethoxycarbonyl); 4-cyanoPhe (4-cyanophenylalanine); p-cyanoPhe (p-cyanophenylalanine); DMSO (dimethylsulfoxide); HFIP (hexafluoroisopropanol).

### Example I

### P-Cyanophenylalanine as an Intrinsic Probe of Amyloid Formation

As noted above, amyloid formation, in vitro, is traditionally followed using fluorescence detected in thioflavin binding experiments. The fluorescence of the dye significantly increases upon binding to the amyloid fibril. The assay, although simple to execute, suffers from some noticeable drawbacks. First, the dye does not bind to amyloid intermediates and thus cannot be used to follow their formation. Second, some compounds can bind to amyloid fibrils, displacing bound thioflavin-T, without inhibiting amyloid formation. In these cases, thioflavin-T assays lead to the incorrect conclusion that the compound is an amyloid inhibitor. Third, the dye is an extrinsic probe. Accordingly, there is always the risk that the apparent kinetics of amyloid assembly could be confounded by the actual kinetics of thioflavin-T binding.

In principle, intrinsic protein fluorescence could be used to follow amyloid formation since tryptophan fluorescence is sensitive to changes in its molecular environment. However, a surprising number of important amyloidogenic polypeptides lack tryptophan including Aβ, α-synuclein and IAPP (amylin), the causative agents of amyloid formation in Alzheimer's disease, Parkinson's disease and type-2 diabetes respectively. Tyrosine fluorescence, as introduced via single amino acid substitution or from naturally occurring tyrosine, might be useful, but it is less sensitive than tryptophan fluorescence and its interpretation is much less straightforward. The change in Tyr quantum yield is not as well understood as that of Trp and it is hard to predict how a change in protein conformation will affect it: there are examples where there is no change in Tyr fluorescence, a decrease in fluorescence, or an increase in fluorescence when proteins go from their unfolded to folded forms as reported in Sato and Raleigh Journal of Molecular Biology 318, 571-582 (2002) hereby incorporated by reference. Furthermore, the time course of changes in the Tyr fluorescence of some amyloid forming proteins does not follow the development of amyloid and in some case there is no significant change in tyrosine fluorescence upon amyloid formation as exemplified in Maji et al Biochemistry 44, 13365-13376 (2005) hereby incorporated by reference It would clearly be desirable to have access to another fluorescent amino acid that could be used as a probe of amyloid formation.

While applicants will not be bound by any theoretical explanation of why embodiments of the present invention work, their reasoning in searching for such a fluorescent amino acid may be instructive. According to that reasoning, an ideal amino acid analog would exhibit a large, easily interpretable change in fluorescence during the process of amyloid formation, but represent only a small perturbation of the structure and hydrophobicity of one or more of the twenty genetically encoded residues, allowing for conservative substitution. p-Cyanophenyalanine (p-cyanoPhe) meets these requirements. Its fluorescence quantum yield is very sensitive to solvent interactions and is decreased significantly in a hydrophobic environment compared to its value in water, making it a sensitive probe of the local environment as described in Tucker et al. (2006) Biopolymers 83, 571-576 (2006), and Aprilakis et al. (2007) Biochemistry 46, 12308-12313, both of which are incorporated by reference. Importantly, its blue-shifted absorption band allows its fluorescence to be selectively excited in the presence of tyrosine or tryptophan. The cyano group is a hydrogen bond acceptor but it has the very desirable feature that it is readily accommodated in the hydrophobic core of proteins since its polarity is intermediate between that of an amide and a methylene. It is also considerably smaller than tryptophan, which minimizes potential property changes upon its substitution for phenylalanine or tyrosine.

In this example, we demonstrated the use of p-cyanoPhe fluorescence to probe amyloid formation using islet amyloid polypeptide (IAPP, amylin) as a test case. IAPP is the principal building block for the formation of pancreatic islet amyloid in type II diabetes. It is thought that Islet amyloid formation plays a role in the pathology of the disease by killing pancreatic β-cells, which contributes to the loss of β-cell mass with a concomitant decline in insulin secretion as described in Westermark et al., Proceedings of the National Academy of Sciences USA 84, 3881-3885 (1987), and Marzban et al., Diabetes 53, 141-148 (2004), both of which are hereby incorporated by reference.

The experimental results demonstrate the utility of p-cyanophenylalanine fluorescence as a probe of IAPP fibrillization and provide new insight into amyloid formation by IAPP. The data here shows that burial of the C-terminal aromatic side chain does not occur during the lag phase, but rather occurs concomitantly with fibril assembly during the growth phase. The data presented here also shows that the C-terminal side chain is partially sequestered from solvent.

A number of important amyloidogenic polypeptides contain phenylalanine and/or tyrosine but lack tryptophan. These include Aβ₁₋₄₂, Aβ₁₋₄₀, calcitonin, insulin and α-synuclein. Thus, p-cyanophenylalanine substitutions are expected to be a generally useful approach to probe amyloid formation especially considering that the derivative can be readily incorporated into proteins by chemical synthesis or by recombinant methods as described in Schultz et al Journal of the American Chemical Society 128, 13984-13985 (2006) incorporated herein by reference. The IAPP analog described here should be a useful reagent for studies of inhibitors of amyloid formation since it avoids the problems associated with the use of extrinsic probes such as dyes as described in LeVine, Amyloid: International Journal of Experimental and Clinical Investigation 2, 1-6 (1995) incorporated herein by reference.

Wild-type IAPP, IAPP-Y37F_{C≡N} and a G-F_{CN}-A-A tetrapeptide were synthesized and the disulfide bond in IAPP was formed using dimethyl sulfoxide (DMSO) based oxidation as described in Abedini et al., Analytical Biochemistry 351, 181-186 (2006) incorporated herein by reference. The synthesis of peptides is well known in the art. Synthesizers such as the Applied Biosystems 433A are commercially available with ample instructions for use and are generally accessible to practitioners in the art. Fmoc-4-cyanoPhe was obtained from NovaBiochem. Peptides were purified via reverse phase HPLC and the identities confirmed by MALDI mass spectroscopy. Thioflavin-T fluorescence experiments were performed with a Jobin Yvon Horiba fluorescence spectrophotometer or with an Applied Phototechnology fluorescence spectrophotometer. P-cyanoPhe fluorescence was excited at 240 nm and detected at 296 nm with instrument slit widths of 10 nm on an Applied Photon Technologies fluorimeter. Stock solutions of IAPP in HFIP (1.58 mM) were prepared as described in Abedini et al., Biochemistry 44, 16284-16291 (2005), hereby incorporated by reference.

Fibrillization reactions were initiated by diluting the stock 50-fold into buffered (20 mM Tris-HCl, pH 7.4) aqueous solution. Final conditions were 32 μM IAPP, 20 mM Tris-HCl, 2% HFIP at pH 7.4. When present, thioflavin-T was at 32 μM. TEM was performed at the University Microscopy Imaging Center at the State University of New York at Stony Brook. 4 μL samples from the wild type and Y37F_{C≡N} IAPP reaction solutions were placed on a carbon-coated 300-mesh copper grid and negatively stained with saturated uranyl acetate. Far-UV CD experiments were performed at 25° C. on an Aviv 62A DS CD spectrophotometer. For far-UV CD wavelength scans, an aliquot from the peptide stock was diluted into 20 mM Tris-HCl buffer at pH 7.4, for a total volume of 250 μL. The final peptide concentration was 0.1 mg/mL in 20 mM Tris-HCl buffer. The spectrum is the average of three repeats in a 0.1 cm quartz cuvette and recorded over a range of 190-250 nm, at 1 nm intervals, with an averaging time of three seconds per scan. Finally, a background spectrum was subtracted from the collected data.

hIAPP is 37 residues in length, contains a disulfide bond linking residues 2 and 7 and has an amidated C-terminus. It contains no Trp, but does have two Phe's (at positions 15 and 23) and a single Tyr at its C-terminus. Tyr-37 was replaced with p-cyanoPhe. The peptide is denoted hIAPP-Y37F_{C≡N}. The sequence of the wild-type human peptide, denoted here hIAPP, is:

KCNTATCATQRLANFLVHSSNNFGAILSSTNVGSNTY

In vitro assays of amyloid formation often involve solubilizing the peptide of interest in a fluorinated alcohol, typically hexafluoroisopropanol (HFIP). The fibrillization reaction is initiated by diluting the stock solution into aqueous buffer. P-CyanoPhe fluorescence is sensitive to the hydrogen bonding properties and polarity of the solvent, thus it is important to test whether or not this protocol significantly affects its fluorescence. A small, soluble p-cyanophenylalanine containing peptide (Gly-Phe_{C≡N}-Ala-Ala) was synthesized for the control studies. The fluorescence intensity of the peptide was the same in water, 2% HFIP and was very similar in 100% HFIP, indicating that there are no problems associated with standard fibrillization protocols. 4-cyanophenylalanine fluorescence is quenched by chloride ion and many biological buffers are made from chloride salts, thus we tested if 20 mM Tris-HCl significantly affected the fluorescence. The fluorescence intensity was reduced by approximately 30%. This does not present any significant problems since the fluorescence of p-cyanophenylalanine decreases much more if it is buried in a hydrophobic environment.

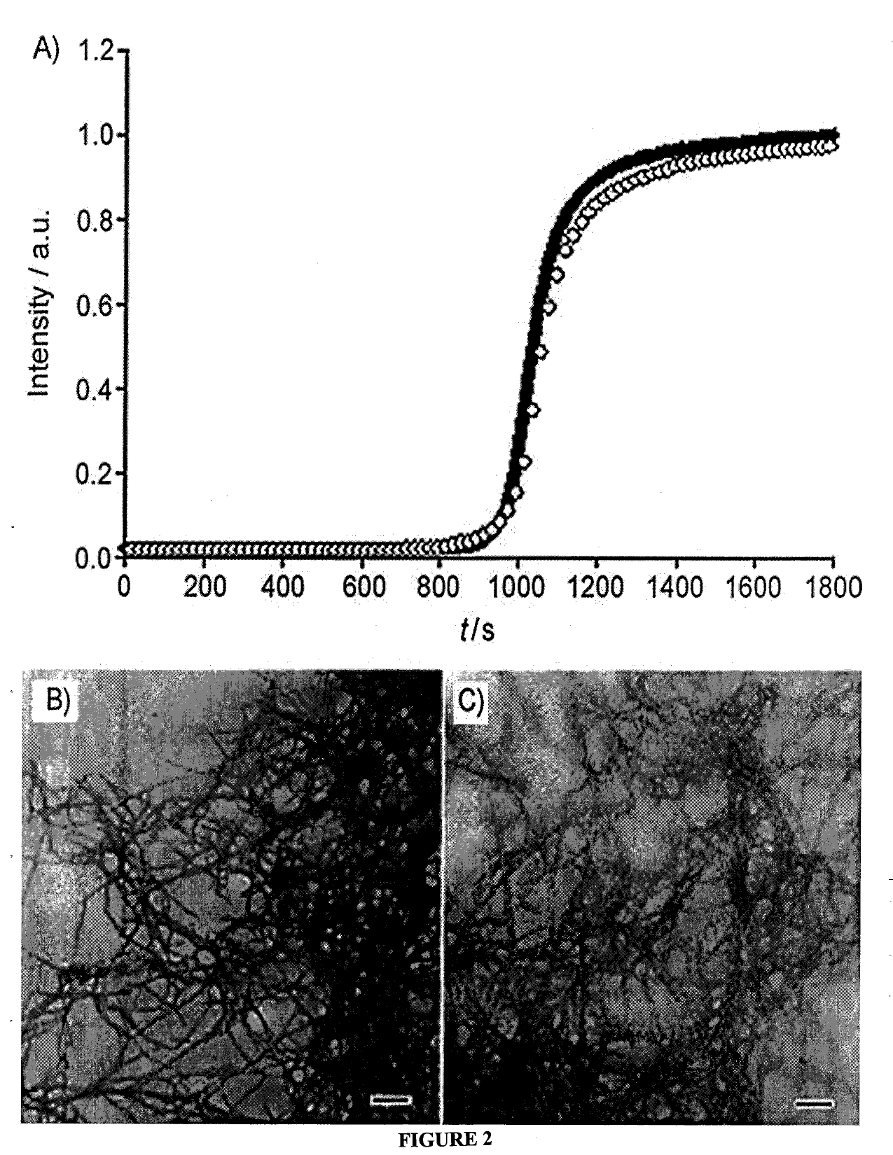

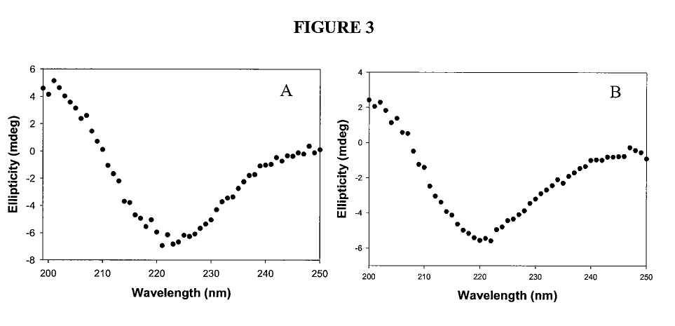

The time course of amyloid formation for wild-type and hIAPP-Y37F_{C≡N} was then compared in order to test if the replacement of the phenolic OH group by a cyano group had a significant effect. Standard thioflavin-T kinetic assays demonstrate that the time course of fibril formation by wild-type hIAPP and hIAPP-Y37F_{C≡N} are essentially identical as shown in FIG. 2A. Quantitative analysis of the data shows thioflavin-T fluorescence at the end of the amyloid formation reaction indicates that the p-cyanophenylalanine substitution does not significantly affect the morphology of the fibrils. This is confirmed by transmission electron microscopy (TEM) and circular dichroism (CD). TEM images of hIAPP and hIAPP-Y37F_{C≡N} are indistinguishable as shown in FIGS. 2B and 2C. CD spectra of hIAPP and hIAPP-Y37F_{C≡N} are also identical as shown in FIGS. 3A and 3B. The kinetic, spectroscopic and TEM studies all demonstrate that the p-cyanoPhe substitution for Tyr is indeed very conservative.

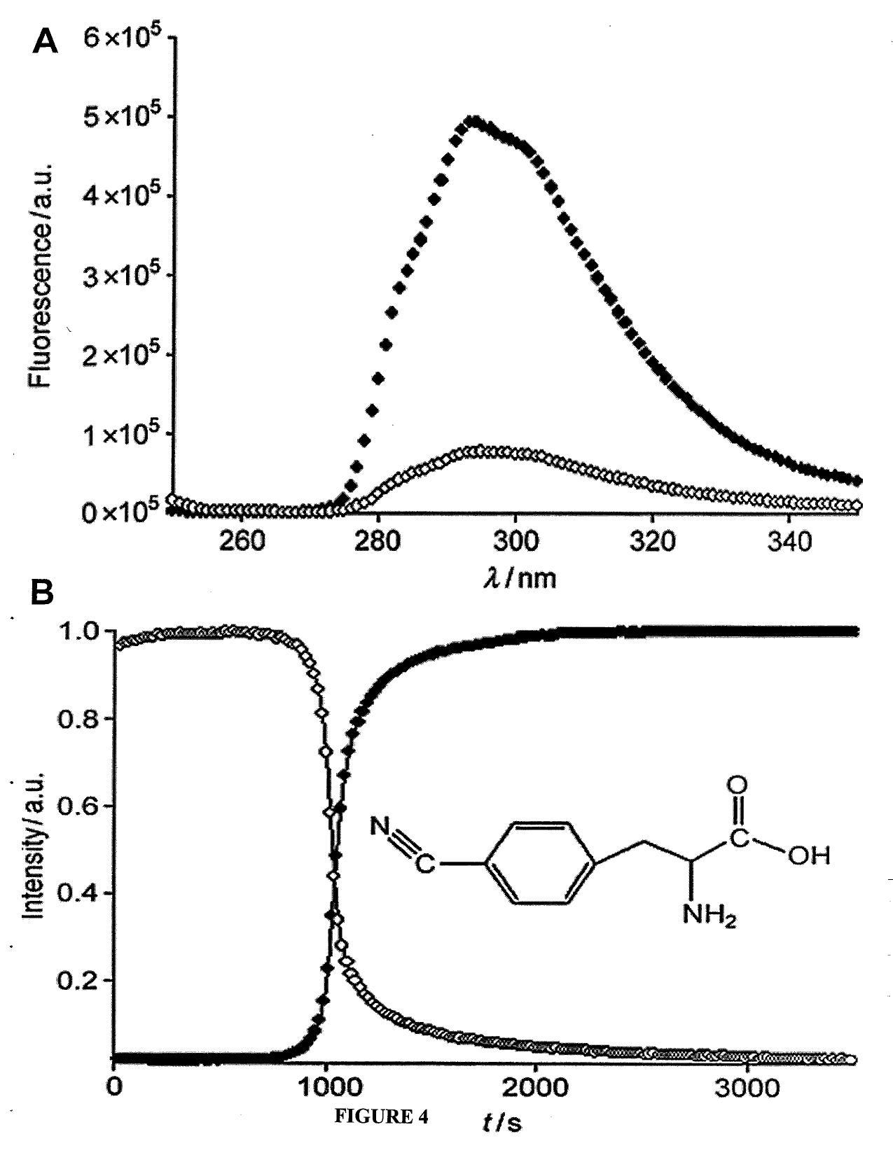

Having confirmed that hIAPP-Y37F_{C≡N} forms amyloid deposits that are similar to wild-type, kinetic investigations using p-cyanophenylalanine fluorescence were performed. There is a large change in fluorescence between the soluble form of the peptide and the fibril form (FIG. 4A). FIG. 4B compares the time course monitored using p-cyanophenylalanine fluorescence to the time course monitored using thioflavin fluorescence. The same stock solution and the same cuvette were used for both measurements. This is important because the time course of amyloid formation is sensitive to the shape of the cuvette and volume of solution used. The ability to conduct experiments under absolutely identical conditions on the same instrument is a key advantage. The plot of thioflavin-T fluorescence vs. time shows the characteristic sigmoidal curve observed in studies of amyloid formation. The time course of the p-cyanophenylalanine fluorescence is identical to that observed for the thioflavin-T experiment. The midpoints (t₅₀) of the two experiments are identical: 1,100±100 s. as determined by p-cyanophenylalanine fluorescence and 1,130±100 s. from thioflavin-T fluorescence. For comparison the midpoints for amyloid formation by wild-type hIAPP is 960±60 s. as determined by thioflavine-T fluorescence. Likewise, the times of the growth period, defined as the time required for the reaction to go from 20 to 80% completion, determined by the two methods are the same: 248±12 s. as determined by p-cyanophenylalanine fluorescence and 247±12 s from thioflavin-T fluorescence.

hIAPP-Y37F_{C≡N} can be mixed with wildtype hIAPP and the p-cyanophenyalanine fluorescence can be used to follow amyloid formation of the mixture. An experiment in which 10% by weight hIAPP-Y37F_{C≡N} was mixed with wildtype hIAPP shows that the time course of amyloid formation as monitored by changes in thioflavin-T fluorescence is identical to the time course monitored by changes in p-cyanophenyalanine fluorescence.

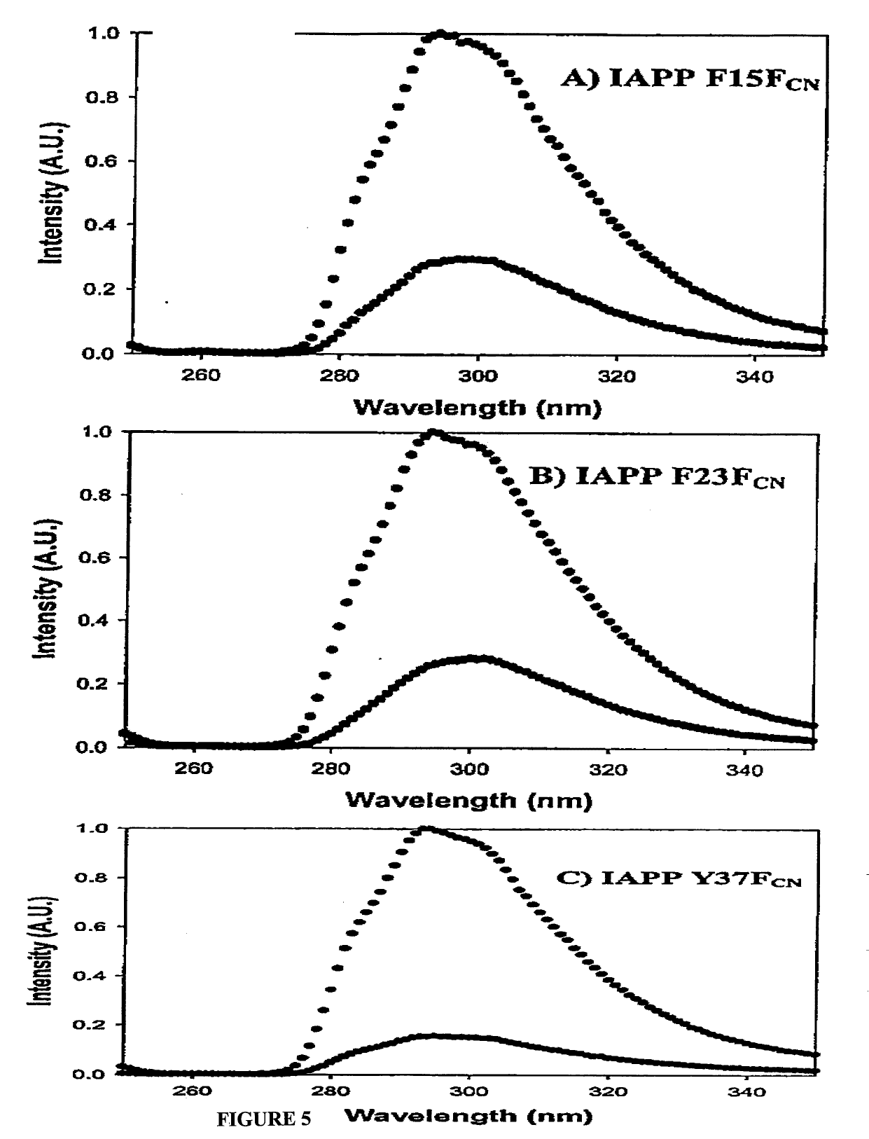

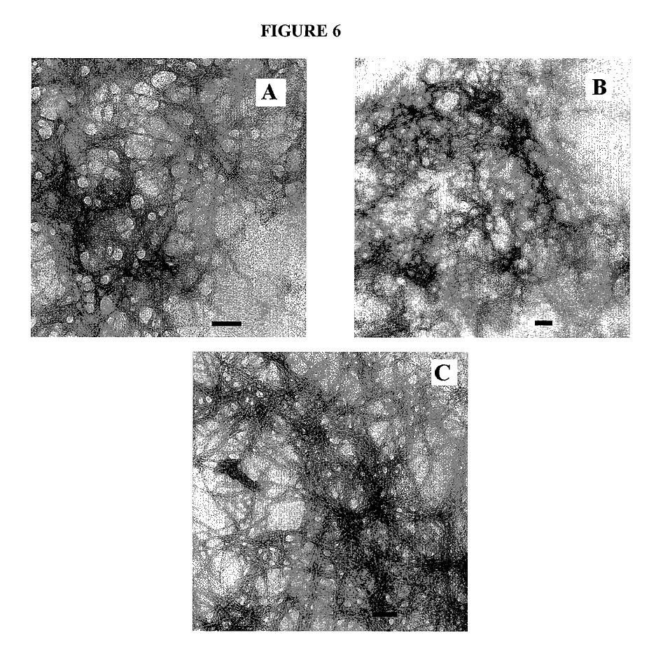

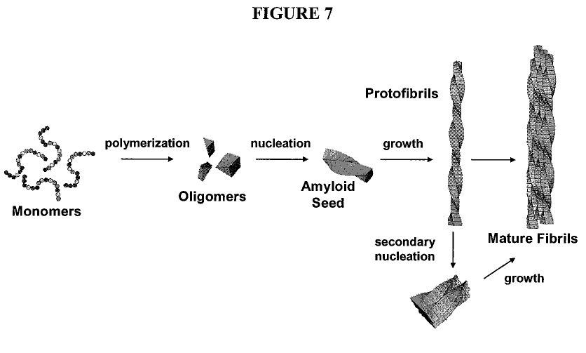

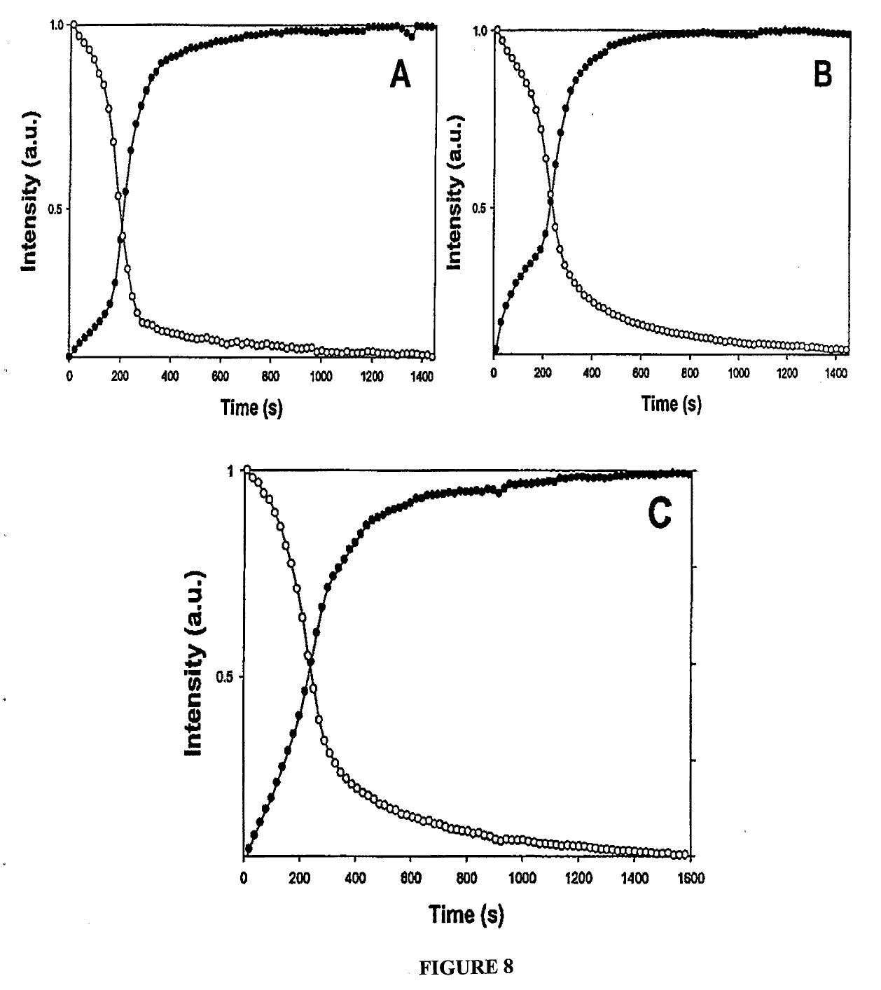

p-Cyanophenyalanine can also be incorporated at position-15 (denoted hIAPP-F15F_{C≡N} or at position-23 of hIAPP (hIAPP-F15F_{C≡N}) and its intrinsic fluorescence used to follow amyloid formation. There is a significant change in p-cyanophenylalanine fluorescence when either hIAPP-F15F_{C≡N} or hIAPP-F23F_{C≡N} forms amyloid as shown in FIGS. 5A, 5B and 5C. The TEM imagines shown in FIGS. 6A, 6B and 6C indicate that the morphology of the amyloid deposits formed by hIAPP-F15F_{C≡N} and hIAPP-F15F_{C≡N} are the same as those formed by hIAPP and hIAPP-Y37F_{C≡N}. The plots of thioflavin-T fluorescence vs. time as shown in FIGS. 8A, 8B and 8C, all show the characteristic sigmoidal curve observed in studies of amyloid formation for both hIAPP-F15F_{C≡N} and hIAPP-F15F_{C≡N}. Furthermore the time course of the p-cyanophenylalanine fluorescence is identical to that observed for the thioflavin-T experiment, demonstrating that p-cyanophenylalanine fluorescence accurately reports on the rate of amyloid formation by both hIAPP-F15F_{C≡N} and hIAPP-F15F_{C≡N}. The midpoints (t₅₀) of the amyloid formation are identical for hIAPP-F15F_{C≡N} and hIAPP-F23F_{C≡N}. The value for hIAPP-F23F_{C≡N} is 972±41 s as determined by thioflavin-T fluorescence and 982±41 s as determined by p-cyanophenyalanine fluorescence. The value for hIAPP-F15F_{C≡N} is 913±10 s as determined by thioflavin-T fluorescence and 936≡10 s as determined by p-cyanophenyalanine fluorescence.

### Example II

### Rifampicin Interferes with Thioflavin-T Assays but does not Prevent Amyloid Formation by IAPP and p-CyanoPhe IAPP Overcomes These Limitations

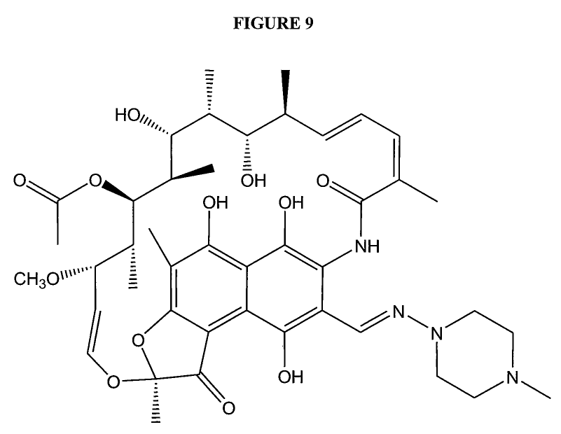

Epidemiological investigations have shown that leprosy patients have a statistically lower probability of senile dementia provided they had been treated with rifampicin or dapsone, suggesting that the drug might prevent Aβ amyloid formation as described in Namba et al., Lancet 340, 978 (1992), and Chui et al., American Journal of Pathology 145, 771-775 (1994), both of which are incorporated by reference. In vitro studies showed that rifampicin did indeed inhibit fibril formation by Aβ₁₋₄₀ and reduced its toxicity to cultured rat PCL2 cells as described in Tomiyama et al., B. B. R. C. 204, 76-83 (1994), hereby incorporated by reference. Rifampicin has been reported to inhibit in vitro amyloid formation by a number of proteins as described in Meier et al., American Journal of Physiology 291, E1317-E1324 (2006), hereby incorporated by reference. More recently, rifampicin has been used to probe the mechanism of IAPP-induced cell death as described in Meier et al. However, the IAPP literature is confusing since some reports argue that rifampicin is an inhibitor of fibril formation and does not inhibit toxicity while others claim that it does not inhibit amyloid formation as described in Meier et al. and Tomiyama et al., Biochemical Journal 322, 859-865 (1997), incorporated herein by reference. Further complicating the issue, Fink and colleagues showed that rifampicin inhibits fibrillzation of a-synuclein but found that an oxidation product of the drug is actually the most potent inhibitory compound as described in Li et al., Chemistry & Biology 11, 1513-1521 (2004), incorporated herein by reference. The conflicting reports on the effects of rifampicin on IAPP fibril formation and their importance for studies of the origin of cellular toxicity prompted us to reexamine the effects of the drug on the in vitro fibrillization of IAPP using transmission electron microscopy (TEM) and fluorescence detected thioflavin-T binding assays and fluorescence p-cyanoPhe studies with IAPPY37F_{C≡N}. The molecular structure of rifampicin is shown in FIG. 9. We show that rifampicin does not prevent amyloid fibril formation by IAPP and does not disaggregate preformed IAPP amyloid but does interfere with thioflavin-T assays. We show that incorporation of p-cyanophenylalanine into hIAPP overcomes these problems.

Reagents. Rifampicin was purchased from Sigma, ascorbic acid was purchased from Fisher Scientific and Thioflavin-T from Aldrich Chemical Company.

Peptide synthesis and purification. Peptides were synthesized on a 0.25 mmol scale using an applied Biosystems 433A peptide synthesizer, using 9-fluornylmethoxycarbonyl (Fmoc) chemistry as described in Abedini et al. (2005) Organic Letters 7, 693-696 (2005), hereby incorporated by reference. Pseudoprolines were incorporated to facilitate the synthesis. The 5-(4′-Fmoc-aminomethyl-3′,5-dimethoxyphenol) valeric acid (PAL-PEG) resin was used to afford an amidated C-terminal. Standard Fmoc reaction cycles were used. The first residue attached to the resin, P-branched residues, residues directly following β-branched residues and pseudoprolines were double coupled. Crude peptides were oxidized by dimethyl sulfoxide (DMSO) for 24 hours at room temperature as described in Abedini et al., Analytical Biochemistry 351, 181-186 (2006), hereby incorporated by reference. The peptides were purified by reverse-phase HPLC using a Vydac C18 preparative column. Analytical HPLC was used to check the purity of the peptides before each experiment. The identity of the pure peptides was confirmed by mass spectrometry using a Bruker MALDI-TOF MS.

Sample Preparation. A 1.58 mM peptide solution was prepared in 100% hexafluoroisopropanol (HFIP) or DMSO and stored at −20° C. A 20 mM rifampicin stock solution was prepared by dissolving rifampicin in DMSO. For the antioxidant experiments, ascorbic acid was dissolved in Tris-HCl buffer and adjusted to pH 7.4 to give a 100 mM ascorbic acid stock solution. The stability of rifampicin in Tris-HCl buffer and ascorbic acid solutions was tested by UV-Vis absorbance at 483 nm as described in Furesz, S., Antibiotica et Chemotherapia 16, 316-351 (1970), hereby incorporated by reference.

Thioflavin-T Fluorescence. All fluorescence experiments were performed with a Jobin Yvon Horiba fluorescence spectrophotometer or with an Applied Phototechnology Fluorescence Spectrophotometer. An excitation wavelength of 450 nm and emission wavelength of 485 nm was used for the thioflavin-T studies. The excitation and emission slits were set at 5 nm. A 1.0 cm cuvette was used and each point was averaged for 1 minute. All solutions for these studies were prepared by diluting filtered stock solution (0.45 μm filter) into a Tris-HCl buffer (pH 7.4) and thioflavin-T solution immediately before the measurement. The final concentration was 32 μM peptide and 25 μM thioflavin-T with or without rifampicin in 2% HFIP for all experiments. The final concentration of ascorbic acid used in antioxidant experiments was 83 μM. All solutions were stirred during the fluorescence experiments. P-cyanophenylalanine fluorescence was excited at 240 nm and detected at 296 nm, with both excitation and emission slits of 10 nm.

Transmission Electron Microscopy (TEM). TEM was performed at the Life Science Microscopy Center at the State University of New York at Stony Brook. The same solutions that were used for the fluorescence measurements were used so that samples could be compared under as similar conditions as possible. 15 μL of peptide solution was placed on a carbon-coated Formvar 200 mesh copper grid for 1 min and then negatively stained with saturated uranyl acetate for 1 min.

Rifampicin interferes with thioflavin-T assays but does not prevent amyloid formation by IAPP. We monitored the apparent time course of fibril formation of IAPP in the presence and in the absence of rifampicin using thioflavin-T assays. Thioflavin-T is a small dye molecule that has proven enormously useful in studies of amyloid formation. Thioflavin-T has a low fluorescence quantum yield in solution which increases significantly when bound to fibrils as described in LeVine, Amyloid: International Journal of Experimental and Clinical Investigation 2, 1-6 (1995), incorporated herein by reference. There is no structure of thioflavin-T bound to any amyloid fibrils but the dye is believed to bind to grooves on the surface of amyloid fibrils. Amyloid is made up of a cross-P structure in which individual β-strands are aligned perpendicular to the fibril axis. In such a structure, side chains in consecutive strands will form a ridge and a set of side chains at positions n and n+2 will lead to two ridges separated by a groove. These grooves are the likely binding sites.

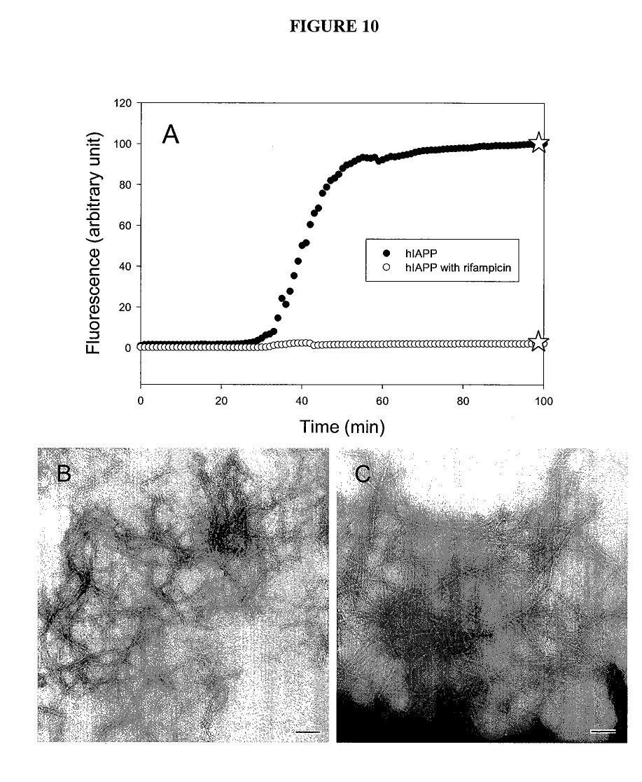

Samples were 32 μM in IAPP and contained either no rifampicin or 15 μM rifampicin. The ratio of drug to IAPP is higher than that reported to inhibit IAPP fibrillization as described in Meier et al. The data collected in the absence of rifampicin is typical of IAPP fibrillization experiments. A lag phase is observed followed by a growth phase with a rapid change in thioflavin-T fluorescence leading to a final plateau where the bound thioflavin-T fluorescence reaches a steady state value as shown in FIG. 10A. TEM images collected of samples corresponding to the end point of the reaction display the classic features of amyloid as shown in FIG. 10B. The results of the experiment in the presence of rifampicin are strikingly different. No significant change in thioflavin-T fluorescence is observed over the entire time course of the reaction as shown in FIG. 10A. Taken alone, the thioflavin-T fluorescence experiment would argue that rifampicin is a potent inhibitor of fibrillization. However, the drug could instead be a competitive inhibitor of thioflavin-T binding to IAPP fibrils or it might quench the fluorescence of the bound thioflavin-T without preventing amyloid formation. Consequently, we recorded TEM images of aliquots of each reaction mixture collected at a time point corresponding to 100 minutes after initiation of the reaction. This is much longer than the time required for IAPP to form amyloid fibrils. The images are displayed in FIGS. 10B and 10C and reveal numerous fibrils with the classic morphology associated with in vitro IAPP fibrils for both samples, indicating that rifampicin did not prevent amyloid formation by IAPP. The key conclusion is that rifampicin, when present at the start of the fibrillization reaction, leads to false negatives in the thioflavin-T assay.

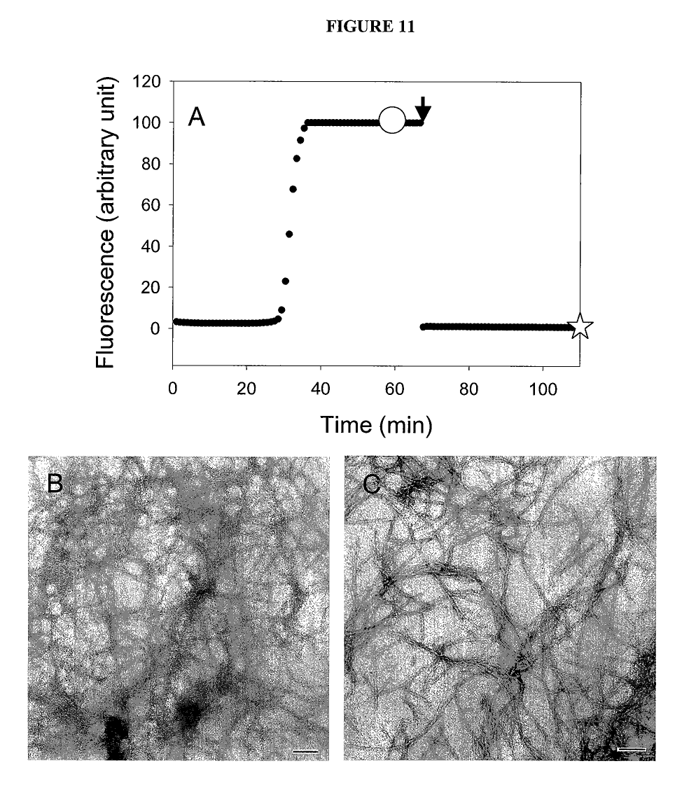

We further investigated the effect of adding rifampicin to preformed fibrils. Such an experiment is often performed to test a compound's ability to disaggregate fibrils. In this case, a compound that eliminated the fluorescence of bound thioflavin-T but did not dissociate fibrils would be incorrectly scored as having the ability to disaggregate fibrils, i.e. it would generate a false positive. It is possible that rifampicin only interferes with thioflavin-T binding to IAPP if it is present in the initial reaction mixture. This seems unlikely since the dye generally does not bind to species populated in the lag phase as described in LeVine, Amyloid: International Journal of Experimental and Clinical Investigation 2, 1-6 (1995), incorporated herein by reference but it is important to test. FIG. 11A displays the result of an experiment in which rifampicin is added to the plateau region of the reaction as indicated by the arrow. Addition of the drug leads to a rapid loss of thioflavin-T fluorescence as shown in FIG. 11A. TEM images of a sample collected before the drug is added are shown in FIG. 11B and confirm that abundant fibrils had formed. Strikingly TEM images collected after the drug was added also display numerous amyloid fibrils as shown in FIG. 11C. Once again, the drug interferes with the thioflavin-T fluorescence response but does not disaggregate IAPP fibrils.

Rifampicin does not prevent IAPP amyloidformation in the presence of antioxidants. The naphthohydroquinone ring in rifampicin is easily oxidized to the quinone form and aqueous solutions of rifampicin are not stable. Under basic conditions they break down to the rifampicin quinone form and under acidic conditions, 3-formyl rifampicin is produced as described in Li et al. Chemistry & Biology 11, 1513-1521 (2004), hereby incorporated by reference. The oxidation product is a more potent inhibitor of α-synuclein fibril formation as described in Li et al. Chemistry & Biology 11, 1513-1521 (2004), hereby incorporated by reference. The experiments described in the previous subsection were conducted in the absence of antioxidants, thus rifampicin will be present as a mixture of the oxidized and reduced form. Studies of rifampicin amyloid interactions are typically conducted without antioxidants. We repeated the studies in the presence of an antioxidant to test if our results were dependent on the oxidation state of the drug. We used ascorbic acid as the antioxidant. The breakdown of rifampicin can be easily monitored by following the changes in its absorption spectrum. In particular, the intensity at 483 nm is significantly reduced if a sample of rifampicin is incubated in aqueous buffer at physiological pH due to oxidation. Control experiments showed that no significant change in the absorbance of rifampicin at 483 nm for at least 1000 minutes in the presence of ascorbic acid. In contrast, a steady decrease is observed in the absence of ascorbic acid.

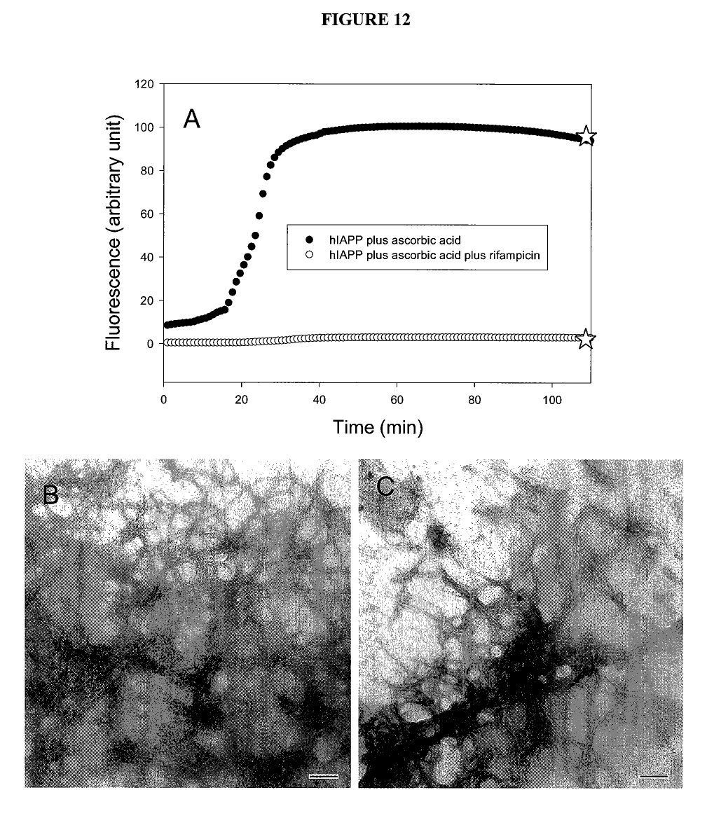

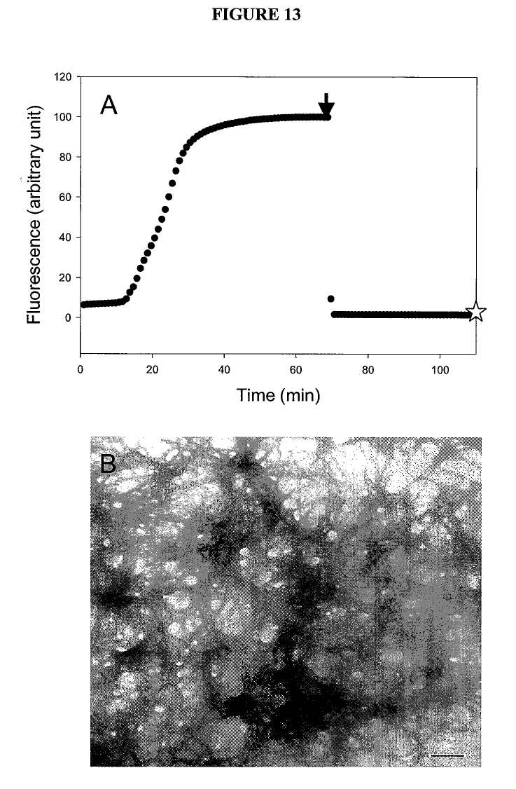

The results are not affected by the presence of the antioxidant; rifampicin still inhibits thioflavin-T fluorescence but does not prevent amyloid formation by IAPP. A sample of IAPP in the presence of 83 μM ascorbic acid yields a typical kinetic curve as monitored by thioflavin-T fluorescence as shown in FIG. 12A, and TEM as shown in FIG. 12B confirms that fibrils were formed. Addition of rifampicin to the reaction mixture at time zero leads to a flat curve with no significant change in the thioflavin-T fluorescence. A sample was removed from this reaction mixture after 100 minutes and TEM images were recorded. They revealed abundant amyloid fibrils as displayed in FIG. 12C. We also repeated the experiment in which rifampicin was added to preformed fibrils. Again we observed the same results in the presence and absence of ascorbic acid. Addition of rifampicin eliminated the thioflavin-T fluorescence but did not dissociate fibrils as shown in FIG. 13A and FIG. 13B.

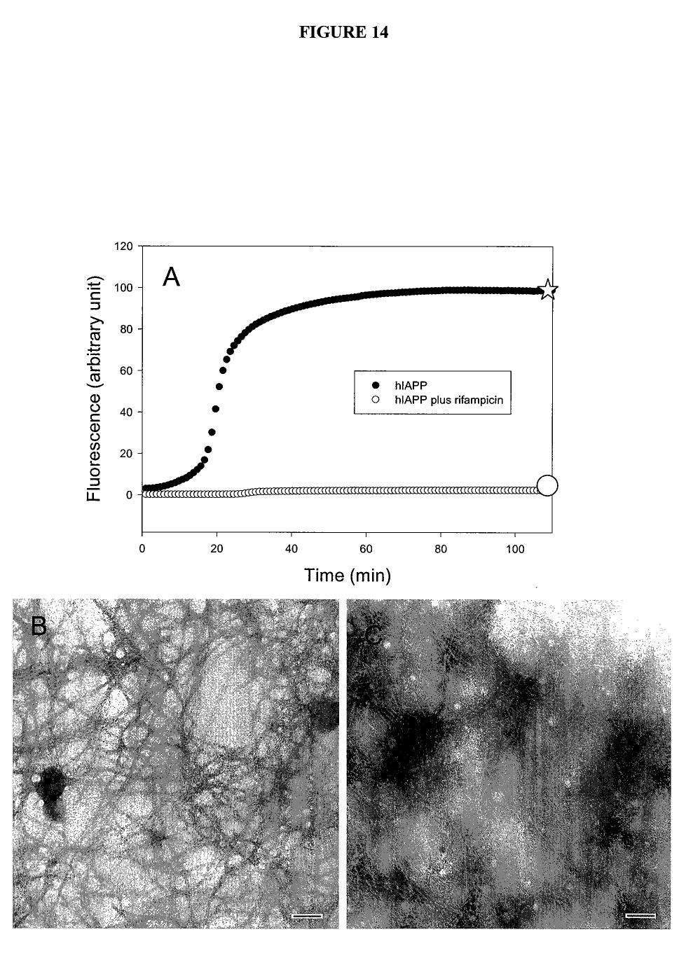

We also examined the effect of oxidized rifampicin by testing samples that had been preincubated in aqueous solutions under conditions that promote oxidation of the drug. A sample of rifampicin was incubated in aqueous solution for 35 days prior to testing its inhibitory potential. No significant difference was observed; the material failed to prevent amyloid formation but did interfere with thioflavin-T fluorescence experiment as shown in FIG. 14.

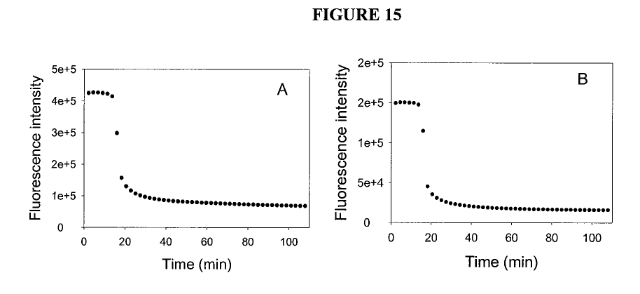

Use of hIAPP-Y37F_{C≡N} allows the kinetics of amyloid formation to be monitored in the presence of rifampicin. FIG. 15 compares the time course of the fluorescence of hIAPP Y37F_{CN} in the presence and absence of rifampicin. The time courses of fibril formation are very similar in the presence and in the absence of the drug (FIG. 15A and FIG. 15B). Quantitative analysis of the data shows that the t₅₀ time (the time for the reaction to reach 50% of the maximum fluorescence intensity) is 19 minutes when rifampicin is present and 18 minutes when it is absent. The respective growth phase, here defined as the time for the reaction to go from 10% to 80% completion are also very similar; 7 minutes in the presence of the drug and 6 minutes in its absence.

The studies described here demonstrated that rifampicin does not prevent amyloid formation by human IAPP, furthermore the results are not an artifact of failing to control the oxidation state of the drug since the same effects were observed in the presence of ascorbic acid and with samples that had been allowed to oxidize for a lengthy incubation time. One important lesson from these studies is that the thioflavin-T based assay can lead to false positives in tests of fibrillization inhibitors. It is impossible to say how general this effect may be but there are likely other small molecules that inhibit thioflavin-T binding or compromise its fluorescence response.

### Example III

An amyloid-forming condition or suspected amyloid-forming condition is established in vitro simply by adding IAPP to a solution as described in the foregoing Examples. Such a condition is established, also, by isolating pancreatic islets from a pancreatic biopsy. Pancreatic biopsies are accomplished, for example, by fine-needle aspiration under radiographic visualization as described in Diederich et al. Cancer Imaging 6, 43-50 (2006), incorporated herein by reference. The islets are maintained in vitro as described in Wang et al., Diabetes 50, 534-539 (2001), incorporated herein by reference. Alternatively, islets are harvested from organ donors and further isolated according, for example, to Warnock et al., Arch. Surg. 140, 735-744 (2005), incorporated herein by reference.

An amyloid-forming condition in vivo is sampled by collecting tissue having an amyloid-forming condition or a suspected amyloid forming condition.

Embodiments of the invention that are directed at monitoring the process of amyloid formation are adapted to screen for agents that inhibit (or aggravate) the process by including in the embodiment a candidate agent and assessing how the agent, preferably over a range of concentrations, affects the process, as determined by fluorescence measurements. The method is straightforward in vitro. The effect of such compounds on an amyloid-forming process that is underway in vivo can be evaluated by monitoring the process, for example, in pancreatic tissue autotransplanted to a superficial region of an animal's body, with anastomoses to the circulation (Arcus et al. (1979) Diabetologia 16:283-346) for blood sampling or to make transcutaneous fluorescence measurements (Shimizu, K. Et al., Applied Optics (2005) 44:2154-2161).

1. A composition selected from the group consisting of:
KCNTATCATQRLANFLVHSSNNFGAILSSTNVGSNTF_{C≡N},
KCNTATCATQRLANFLVHSSNNF^{C≡N}GAILSSTNVGSNTY,
KCNTATCATQRLANF_{C≡N}LVHSSNNFGAILSSTNVGSNTY,
KCNTATCATQRLANFLVHSSNNF_{C≡N}GAILSSTNVGSNTF_{C≡N},
KCNTATCATQRLANF_{C≡N}LVHSSNNF_{C≡N}GAILSSTNVGSNTY,
KCNTATCATQRLANF_{C≡N}LVHSSNNFGAILSSTNVGSNTF_{C≡N}, and
KCNTATCATQRLANF_{C≡N}LVHSSNNF_{C≡N}GAILSSTNVGSNTF_{C≡N}, wherein
F_{C≡N} is p-cyanophenylalanine.

2. A method of monitoring a process of amyloid formation in vitro comprising:
(i) providing
a) a fluorescence spectrometer, and
b) a composition comprising an islet amyloid polypeptide having a p-cyanophenylalanine substitution,
(ii) creating an in vitro condition in which amyloid forms,
(iii) adding said substituted polypeptide to said amyloid-forming condition to make a mixture, and
(iv) detecting a fluorescence from said mixture.

3. The method of claim 2 wherein said mixture is made from an amyloid-forming condition created in a solution comprising said polypeptide.

4. The method of claim 2 wherein said in vitro condition comprises a tissue.

5. The method of claim 2 wherein said in vitro condition comprises a cell.

6. The method of claim 5 wherein said cell comprises a cell culture.

7. The method of claim 6 wherein said cell culture is a primary cell culture.

8. The method of claim 5 wherein said cell is a transformed cell.

9. The method of claim 5 wherein said cell is a stem cell.

10. The method of claim 4 wherein said tissue comprises a tissue culture.

11. The method of claim 4 wherein said tissue is a biopsied tissue.

12. The method of claim 4 wherein said tissue is a tissue, or a sample therefrom, for transplantation.

13. The method of claim 4 wherein said tissue is a mammalian tissue.

14. The method of claim 5 wherein said cell is a mammalian cell.

15. The method of claim 13 wherein said tissue is from a mammal suspected of having type II diabetes.

16. The method of claim 14 wherein said cell is from a mammal suspected of having type II diabetes

17. The method of claim 2 wherein said mixture comprises a test compound.

18. A method of monitoring a process of amyloid formation in an organism in vivo comprising:
(i) providing a fluorescence spectrometer
(ii) administering to said organism an islet amyloid polypeptide having a p-cyanophenylalanine substitution to create a treated organism, and
(iii) detecting a fluorescence in or from said treated organism.

19. The method of claim 18 further comprising administering to said organism a test compound.

20. A method of monitoring a process of amyloid formation comprising:
(i) providing
a) a fluorescence spectrophotometer,
b) a composition comprising an islet amyloid polypeptide having a p-cyanophenylalanine substitution, and
c) a condition in which amyloid forms,
(ii) adding said composition to said condition,
(iii) removing from said condition a sample of said condition, and
(iv) detecting a fluorescence from said sample.

21. A method of monitoring a process of amyloid formation comprising:
(i) providing
a) a fluorescence spectrophotometer,
b) a composition comprising an islet amyloid polypeptide having a p-cyanophenylalanine substitution, and
c) a condition in which amyloid forms,
(ii) adding said composition to said condition, and
(iii) detecting a fluorescence from said condition.

22. The method of claim 20 or 21 wherein said condition is in vitro.

23. The method of claim 20 or 21 wherein said condition is in vivo.
